(19) United States
(12) Patent Application Publication
(10) Pub. No.: US 2015/0148671 A1
(43) Pub. Date: May 28, 2015

(54) Non-invasive Liver Fibrosis Evaluation Device and a Method Thereof

(71) Applicant: NATIONAL TAIWAN UNIVERSITY, Taipei City (TW)

(72) Inventors: Chiung-Nien CHEN, Taipei City (TW); Ming-Chih HO, Taipei City (TW); King-Jen CHANG, Taipei City (TW); Po-Hsiang TSUI, Taipei City (TW); Yu-Chen SHU, Taipei City (TW)

(73) Assignee: NATIONAL TAIWAN UNIVERSITY, Taipei City (TW)

(21) Appl. No.: 14/184,109

(22) Filed: Feb. 19, 2014

(30) Foreign Application Priority Data
Nov. 22, 2013 (TW) 102142707

(51) Int. Cl. A61B 8/08; A61B 8/13

(52) U.S. Cl. CPC A61B 8/085; A61B 8/13; A61B 8/5223; A61B 8/5207
USPC 600/438

(57) ABSTRACT

A non-invasive liver fibrosis evaluation device and a method thereof are related. The device comprises an ultrasound unit, a Nakagami parameter generation unit, a hardness value generation unit, a data base, and a determination unit. The method comprises steps of: scanning the external body part corresponding to the liver by a transducer of the ultrasound unit to produce plural ultrasound image data sets; analyzing one ultrasound image data set with the Nakagami distribution to produce a Nakagami parameter by using the. Nakagami parameter generation unit; analyzing plural ultrasound image data sets to produce a hardness value by using the hardness value generation unit; and evaluating the liver fibrosis by comparing the Nakagami parameter and the hardness value with plural reference parameter sets stored in the data base by using the determination unit.

### FIELD OF THE INVENTION

The present invention relates to a non-invasive liver fibrosis evaluation device and a method thereof, and more particular to a non-invasive liver fibrosis evaluation device and a method thereof using Nakagami distribution and elastogram analysis to process the ultrasound images of liver to obtain Nakagami parameters and hardness values, which are then compared with the reference parameter sets of a data base to evaluate the liver fibrosis stage, so that the early stage of liver cirrhosis can be predicted.

### BACKGROUND OF THE INVENTION

When liver cells are injured, a cascade of degeneration events are induced chronically, which results in hepatic restructuring and ultimately leads to the formation of liver cirrhosis. The progression o the degeneration is gradual and slow. Besides, there is almost no apparent symptom before the outbreak of the disease, and thus it is often neglected in its early stage, so that it is mostly in its late stage when the clinical features of liver cirrhosis are finally presented. Cirrhosis of the liver is an irreversible process. Although the medical treatments for chronic liver disease are continuously under research, there is still no effective treatment to heal a cirrhotic liver. Therefore, early detection is important to the prevention of liver cirrhosis.

Conventional diagnoses for cirrhosis are mostly determined by the medical knowledge and clinical experience of the physicians or by the result of histochemical staining of sections from an invasive liver biopsy. However, patients undergoing liver biopsy must take high risks of the surgery. Therefore, the misdiagnosis rate of liver cirrhosis and the mortality have remained high.

To prevent risks of performing invasive detections, more effective and reliable non-invasive detection methods for clinical use are demanded. Image diagnoses are common non-invasive detection methods at the present time, which includes ultrasonic, X-ray, CT scan, and MRI imaging technologies. Among those imaging technologies, the ultrasonic imaging has the advantages of real-time imaging, high safety, fully portable, and low cost, etc., and therefore it is the most accepted first-line diagnostic imaging for physicians and patients.

Generally, the ultrasonic imaging technology is performed by scanning a biological tissue with ultrasound. An ultrasonic pulse is first transmitted into the tissue by an ultrasound transducer, and plural echo signals are scattered by the scatterers which are randomly distributed in the tissue with complicate scattering mechanism. The signals that are reflected back and received by the transducer are called the backscattering signals. To have more advanced detection of the biological tissue, the ultrasound elastic imaging technology is therefore developed. By compressing the biological tissue first, the randomly distributed scatterers in the tissue displace along the three orthogonal directions, so that the ultrasound echo signals obtained before and after the compression are different. By analyzing the echo signals received with different compression using the strain-stress analysis, the elastic characteristics of the biological tissue can be obtained. By making use of this physical property, the elastic characteristics of a liver scanning area can be obtained by compressing the scanning area when performing the ultrasonic scanning of the liver. However, when only a small portion of liver is fibrous in the early stage of liver cirrhosis, the abovementioned method can not well distinguish the degree of liver lesion, which limits the application of the method in the detection of early stage of liver cirrhosis.

Accordingly, in order to solve the above problem, the present invention to provides a non-invasive liver fibrosis evaluation device and a method thereof, so that the early stage of liver cirrhosis can be predicted.

### SUMMARY OF THE INVENTION

The main object of the present invention is to provide a non-invasive liver fibrosis evaluation device and a method thereof, which use ultrasound technology and image analysis methods to obtain quantitative data (Nakagami parameter and hardness value) of the characteristics of a liver tissue scanning area, and the quantitative data is compared with the reference data sets in a data base to evaluate the liver fibrosis stage.

To reach the object stated above, the present invention provides a non-invasive liver fibrosis evaluation device, which comprises an ultrasound unit, a Nakagami parameter generation unit, a hardness value generation unit, a data base, and a determination unit. The ultrasound unit comprises a transducer for scanning the external body part corresponding to the liver to produce plural ultrasound image data sets. The Nakagami parameter generation unit is used for analyzing one ultrasound image data set with the Nakagami distribution to produce a Nakagami parameter. The hardness value generation unit is used for analyzing plural ultrasound image data sets to produce an elastogram, and then a hardness value is produced by analyzing the elastogram. The data base stores plural reference parameter sets, each of which includes a Nakagami parameter and a hardness value. The determination unit is used for evaluating the liver fibrosis stage by comparing the produced Nakagami parameter and the hardness value with the plural reference parameter sets stored in the data base.

Moreover, the present invention provides a non-invasive liver fibrosis evaluation method, which comprises the following steps:

A1. scanning the external body part corresponding to the liver by a transducer of an ultrasound unit to produce plural ultrasound image data sets;

A2. analyzing one ultrasound image data set with the Nakagami distribution to produce a Nakagami parameter;

A3. analyzing plural ultrasound image data sets to produce an elastogram, and then analyzing the elastogram to produce a hardness value;

A4. providing a data base for storing plural reference parameter sets, each of which comprises a Nakagami parameter and a hardness value; and

A5. evaluating the liver fibrosis stage by comparing the produced Nakagami parameter and the hardness value with the plural reference parameter sets stored in the data base.

In implementation, the plural reference parameter sets of the data base are classified based on the classification of a clinical evaluation of liver fibrosis stage.

In implementation, the plural reference parameter sets of the data base form a two dimensional distribution by using the Nakagami parameter and the hardness value as two independent variables, so that the produced Nakagami parameter and the hardness value are compared with the plural reference parameter sets of the data base by using coordinate analysis.

In implementation, the non-invasive liver fibrosis evaluation device further comprises an index generation unit, which generates an index of liver fibrosis stage after comparing the produced Nakagami parameter and the hardness value with the plural reference parameter sets of the data base.

In implementation, the plural ultrasound image data sets are produced by applying manually generated pressure.

In implementation, the plural ultrasound image data sets are produced by applying acoustic impulses generated by the transducer.

The present invention will be understood more fully by reference to the detailed description of the drawings and the preferred embodiments below.

### BRIEF DESCRIPTION OF DRAWINGS

FIG. 1 is a schematic view showing a non-invasive liver fibrosis evaluation device according to an embodiment of the present invention.

FIG. 2A is a schematic view showing the reference parameter sets of the data base form a two dimensional distribution by using the Nakagami parameter and the hardness value as two independent variables.

FIG. 2B is a schematic view showing a comparison of the produced Nakagami parameter and hardness value to the reference parameter sets of the data base.

FIG. 3 is a flow chart of a non-invasive liver fibrosis evaluation method according to an embodiment of the present invention.

### DETAILED DESCRIPTIONS OF PREFERRED EMBODIMENTS

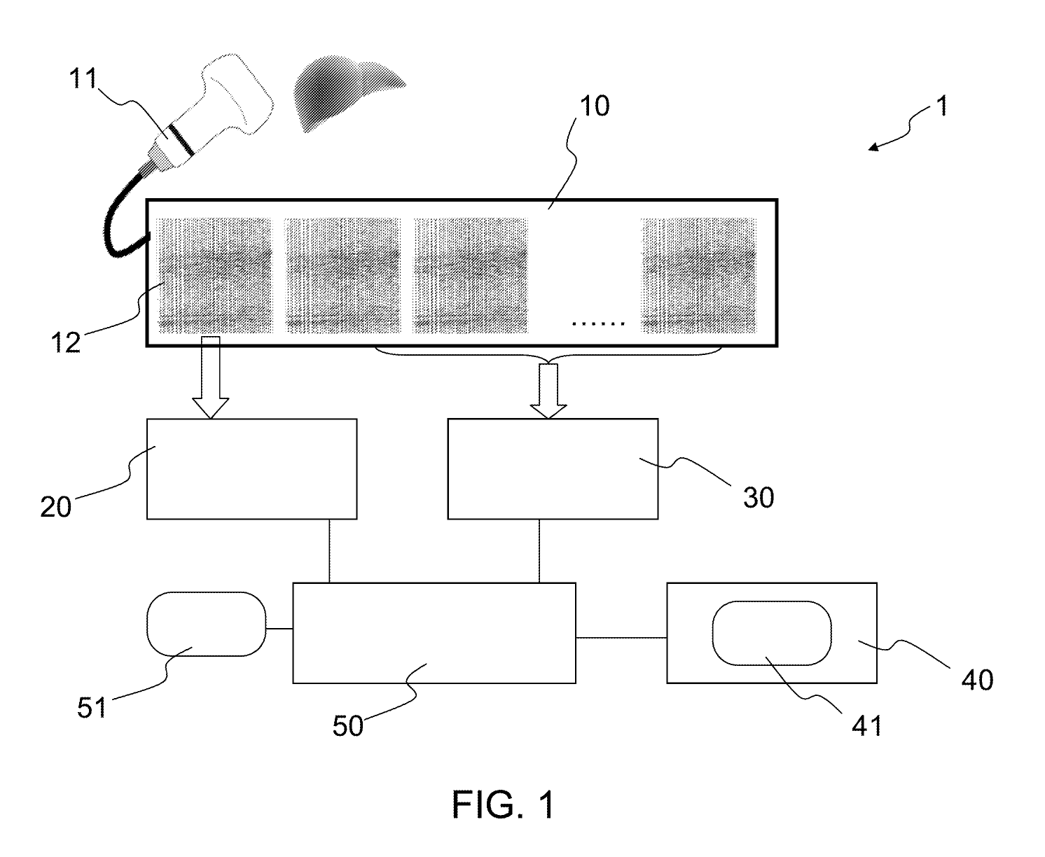

FIG. 1 is a schematic showing a non-invasive liver fibrosis evaluation device 1 according to an embodiment of the present invention. The device 1 comprises an ultrasound unit 10, a Nakagami parameter generation unit 20, a hardness value generation unit 30, a data base 40, and a determination unit 50. The ultrasound unit 10 comprises a transducer 11 for scanning the external body part corresponding to the liver to produce ultrasound image data 12. Plural ultrasound image data sets 12 can be produced by compressing the scanning area with gradual force during the scanning process, so that the elasticity information of the liver tissue of the scanning area can be obtained.

The distribution of the scatterer in liver tissue can be revealed by analyzing the ultrasonic backscattered signals using different statistical distribution models, such as the pre-Rayleigh distribution, the Rayleigh distribution, the post-Rayleigh distribution, or the Nakagami distribution. Comparatively, the Nakagami statistical model has less computational complexity and is general enough to describe a wide range of the statistics of the backscattering envelope images of medical ultrasound, including pre-Rayleigh, Rayleigh, and post-Rayleigh distributions. Therefore, the present invention analyzes the ultrasonic backscattered signals using the Nakagami statistical model. The ultrasonic backscattered signals received by the transducer are demodulated to form an envelope image of the scanned area. The envelope image is then analyzed with the Nakagami distribution to obtain a Nakagami parameter (i.e. the Nakagami-m parameter). The envelope image can be divided into plural windows, and then the Nakagami parameter mᵢ of each window i is calculated. The Nakagami-m parameter can be the average of mᵢ of the whole or a part of the envelope image. In the present invention, the distribution of the scatterer in liver tissue of the scanned area is revealed by analyzing one of the plural ultrasound image data sets 12 using the Nakagami parameter generation unit 20 according to the above theory to obtain a Nakagami-m parameter. The data set used in the above analysis can be any one of the plural ultrasound image data sets 12, in which the ultrasound image data collected before compressing the liver tissue is more preferred.

The hardness value generation unit 30 is used for analyzing plural ultrasound image data sets 12 to produce an elastogram, and then a hardness value is produced by analyzing the elastogram. The elastogram can be produced by using two or more than two of the plural ultrasound image data sets, in which two ultrasound image data sets collected one before and one after compressing the liver tissue are more preferred.

The data base 40 stores plural reference parameter sets 41, each of which includes a Nakagami parameter and a hardness value. The determination unit 50 is used for evaluating the liver fibrosis stage by comparing the produced Nakagami parameter and the hardness value with the plural reference parameter sets 41 stored in the data base 40.

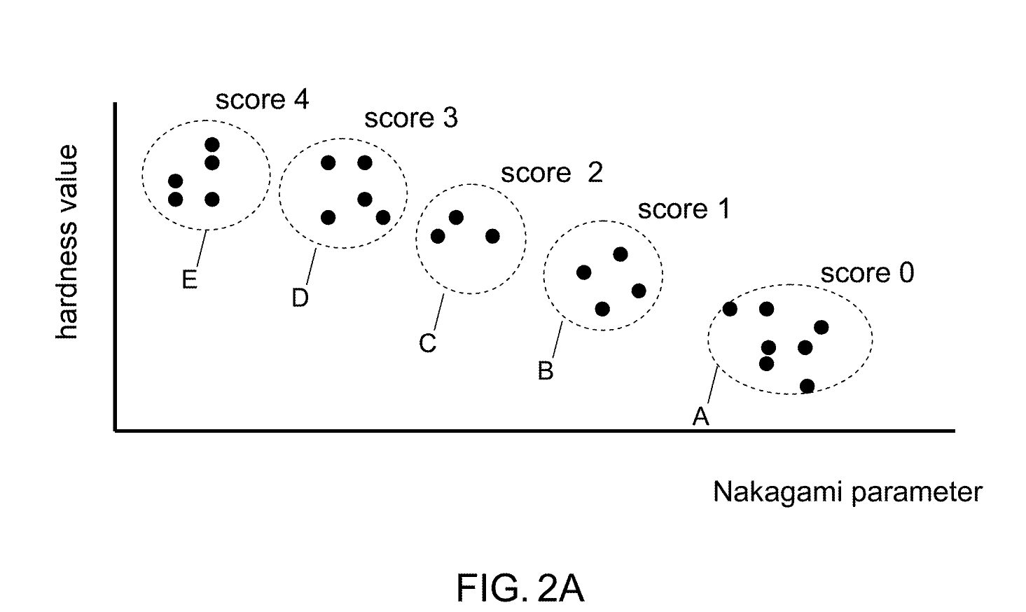

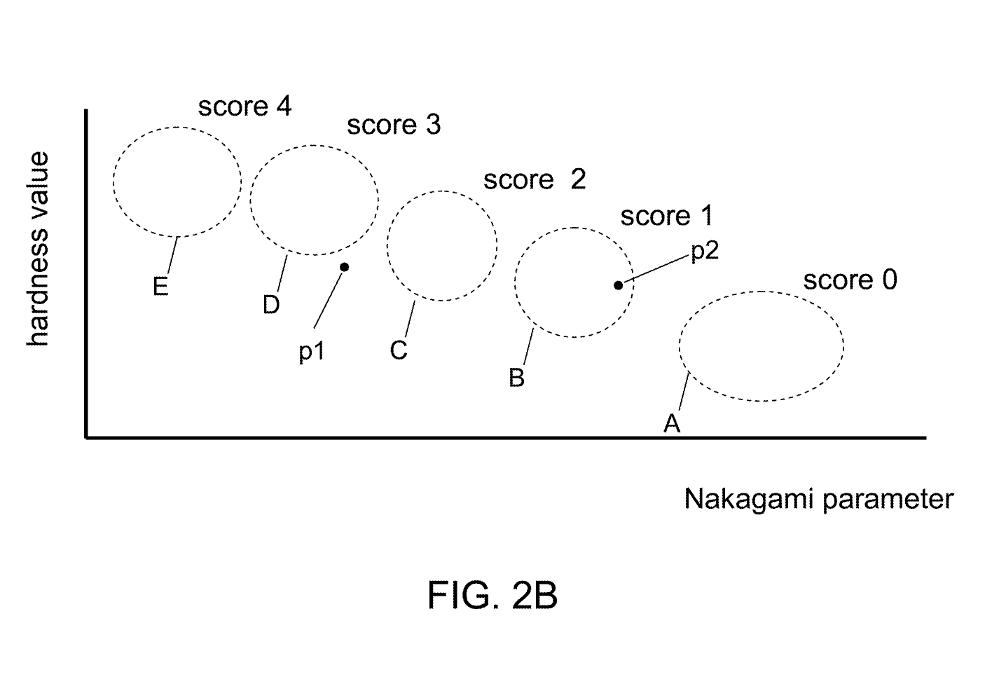

The plural reference parameter sets 41 of the data base 40 are classified based on the classification of a clinical evaluation of liver fibrosis stage. By using the Nakagami parameter and the hardness value as two independent variables, the plural reference parameter sets 41 of the data base 40 form a two dimensional distribution. As shown by the schematic of an embodiment in FIG. 2A, the distribution of the plural reference parameter sets can be divided into five regions denoted by A, B, C, D, and E corresponding respectively to the classification of a clinical evaluation of liver fibrosis stage scored by 0 to 4. The produced Nakagami parameter and the hardness value can be compared with the plural reference parameter sets 41 of the data base 40 by using coordinate analysis. As shown in FIG. 2B, the coordinates of the point of the produced Nakagami parameter and the hardness value is first positioned, and then the one among the five regions which has the shortest distance to the coordinates is calculated. In FIG. 2B, for example, coordinates of point p1 has the shortest distance to region D, and coordinates of point p2 has the shortest distance to region B. The non-invasive liver fibrosis evaluation device 1 may further comprise an index generation unit 51, which generates an index of liver fibrosis stage after comparing the produced Nakagami parameter and the hardness value with the plural reference parameter sets 41 of the data base 40. In the above embodiment, the score of the region having the shortest distance to the coordinates of the point of the produced Nakagami parameter and the hardness value is taken as the score of the point. Accordingly, p1 in the above embodiment has the score of 3, and p2 has the score of 1.

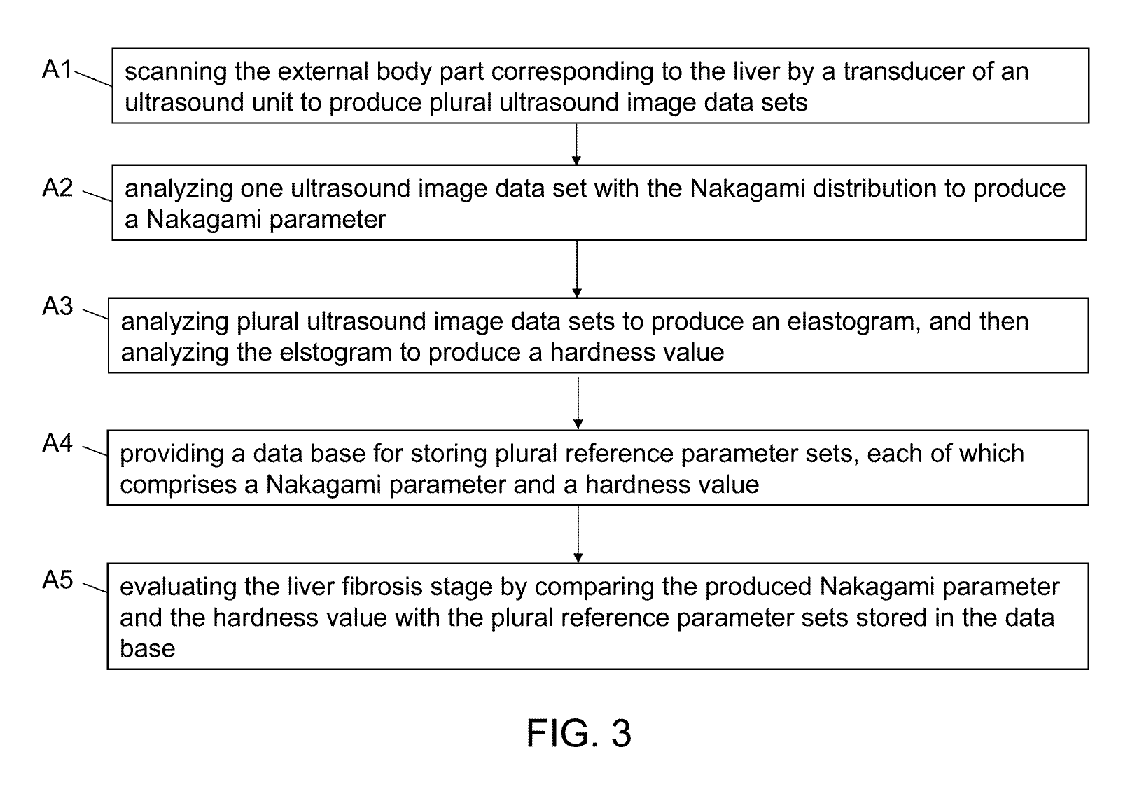

Referring to the flow chart of FIG. 3, the present invention provides a non-invasive liver fibrosis evaluation method, which comprises steps of:

A1. scanning the external body part corresponding to the liver by a transducer 11 of an ultrasound unit 10 to produce plural ultrasound image data sets 12;

A2. analyzing one ultrasound image data set 12 with the Nakagami distribution to produce a Nakagami parameter;

A3. analyzing plural ultrasound image data sets 12 to produce an elastogram, and then analyzing the elastogram to produce a hardness value;

A4. providing a data base 40 for storing plural reference parameter sets 41, each of which comprises a Nakagami parameter and a hardness value; and

A5. evaluating the liver fibrosis stage by comparing the produced Nakagami parameter and the hardness value with the plural reference parameter sets 41 stored in the data base 40.

The data set used in the analysis in step A2 described above can be any one of the plural ultrasound image data sets 12, in which the ultrasound image data collected before compressing the liver tissue is preferable. The data sets used to produced the elastogram in step A3 described above can be two or more than two of the plural ultrasound image data sets, in which two ultrasound image data sets collected one before and one after compressing the liver tissue are preferable.

In step A4 described above, the plural reference parameter sets 41 of the data base 40 can be classified based on the classification of a clinical evaluation of liver fibrosis stage. By using the Nakagami parameter and the hardness value as two independent variables, the plural reference parameter sets 41 of the data base 40 form a two dimensional. In step A5, the produced Nakagami parameter and the hardness value can be compared with the plural reference parameter sets of the data base by using coordinate analysis. An index of liver fibrosis stage may be generated after comparing the produced Nakagami parameter and the hardness value with the plural reference parameter sets of the data base.

In the embodiments of the present invention, the ultrasound unit 10 can include any type of clinical ultrasound instruments. The plural ultrasound image data sets 12 may be produced by applying manually generated pressure through the transducer. The plural ultrasound image data sets 12 may also be produced by applying acoustic impulses generated by the transducer, such as by using the acoustic radiation force impulse imaging (ARFI).

To sum up, the non-invasive liver fibrosis evaluation device and a method thereof provided by the present invention can indeed get its anticipated object to obtain a Nakagami parameter and a hardness value by using the Nakagami distribution and the elastography imaging to analyze the ultrasonic scan images of a liver, and to evaluate the liver fibrosis stage by comparing the produced Nakagami parameter and the hardness value with the reference data sets in a data base, so that the early stage of liver cirrhosis can be effectively predicted as early as possible.

The description referred to the drawings stated above is only for the preferred embodiments of the present invention. Many equivalent local variations and modifications can still be made by those skilled at the field related with the present invention and do not depart from the spirit of the present invention, so they should be regarded to fall into the scope defined by the appended claims.

What is claimed is:

1. A non-invasive liver fibrosis evaluation device, comprising:
an ultrasound unit, comprising a transducer for scanning the external body part corresponding to the liver to produce plural ultrasound image data sets;
a Nakagami parameter generation unit, used for analyzing one ultrasound image data set with the Nakagami distribution to produce a Nakagami parameter;
a hardness value generation unit, used for analyzing plural ultrasound image data sets to produce a hardness value;
a data base, storing plural reference parameter sets, each of which includes a Nakagami parameter and a hardness value; and
a determination unit, used for evaluating the liver fibrosis stage by comparing the produced Nakagami parameter and the hardness value with the plural reference parameter sets stored in the data base.

2. The non-invasive liver fibrosis evaluation device according to claim 1, wherein the plural reference parameter sets of the data base are classified based on the classification of a clinical evaluation of liver fibrosis stage.

3. The non-invasive liver fibrosis evaluation device according to claim 2, wherein the plural reference parameter sets of the data base form a two dimensional distribution by using the Nakagami parameter and the hardness value as two independent variables, so that the produced Nakagami parameter and the hardness value are compared with the plural reference parameter sets of the data base by using coordinate analysis.

4. The non-invasive liver fibrosis evaluation device according to claim 2, further comprising an index generation unit, which generates an index of liver fibrosis stage after comparing the produced Nakagami parameter and the hardness value with the plural reference parameter sets of the data base.

5. The non-invasive liver fibrosis evaluation device according to claim 1, wherein the plural reference parameter sets of the data base form a two dimensional distribution by using the Nakagami parameter and the hardness value as two independent variables, so that the produced Nakagami parameter and the hardness value are compared with the plural reference parameter sets of the data base by using coordinate analysis.

6. The non-invasive liver fibrosis evaluation device according to claim 1, further comprising an index generation unit, which generates an index of liver fibrosis stage after comparing the produced Nakagami parameter and the hardness value with the plural reference parameter sets of the data base.

7. A non-invasive liver fibrosis evaluation method, comprising steps of:
A1. scanning the external body part corresponding to the liver by a transducer of an ultrasound unit to produce plural ultrasound image data sets;
A2. analyzing one ultrasound image data set with the Nakagami distribution to produce a Nakagami parameter;
A3. analyzing plural ultrasound image data sets to produce an elastogram, and then analyzing the elastogram to produce a hardness value;
A4. providing a data base for storing plural reference parameter sets, each of which comprises a Nakagami parameter and a hardness value; and
A5. evaluating the liver fibrosis stage by comparing the produced Nakagami parameter and the hardness value with the plural reference parameter sets stored in the data base.

8. The non-invasive liver fibrosis evaluation method according to claim 7, wherein, in step A4, the plural reference parameter sets of the data base are classified based on the classification of a clinical evaluation of liver fibrosis stage.

9. The non-invasive liver fibrosis evaluation method according to claim 8, wherein, in step A5, the plural reference parameter sets of the data base form a two dimensional distribution by using the Nakagami parameter and the hardness value as two independent variables, so that the produced Nakagami parameter and the hardness value are compared with the plural reference parameter sets of the data base by using coordinate analysis.

10. The non-invasive liver fibrosis evaluation method according to claim 8, wherein, in step A5, an index of liver fibrosis stage is generated after comparing the produced Nakagami parameter and the hardness value with the plural reference parameter sets of the data base.

11. The non-invasive liver fibrosis evaluation method according to claim 8, wherein, in step A1, the plural ultrasound image data sets are produced by applying manually generated pressure.

12. The non-invasive liver fibrosis evaluation method according to claim 8, wherein, in step A1, the plural ultrasound image data sets are produced by applying acoustic impulses generated by the transducer.

13. The non-invasive liver fibrosis evaluation method according to claim 7, wherein, in step A5, the plural reference parameter sets of the data base form a two dimensional distribution by using the Nakagami parameter and the hardness value as two independent variables, so that the produced Nakagami parameter and the hardness value are compared with the plural reference parameter sets of the data base by using coordinate analysis.

14. The non-invasive liver fibrosis evaluation method according to claim 7, wherein, in step A5, an index of liver fibrosis stage is generated after comparing the produced Nakagami parameter and the hardness value with the plural reference parameter sets of the data base.

15. The non-invasive liver fibrosis evaluation method according to claim 7, wherein, in step A1, the plural ultrasound image data sets are produced by applying manually generated pressure.

16. The non-invasive liver fibrosis evaluation method according to claim 7, wherein, in step A1, the plural ultrasound image data sets are produced by applying acoustic impulses generated by the transducer.
